(19) United States
(12) Patent Application Publication
(10) Pub. No.: US 2005/0027309 A1
(43) Pub. Date: Feb. 3, 2005

(54) Guidewire system

(76) Inventor: Samuel Shiber, Manchester, NH (US)

Correspondence Address: SAMUEL SHIBER, 365 KEARNEY CR, MANCHESTER, NH, 03104, US

(21) Appl. No.: 10/937,134

(22) Filed: Sep. 9, 2004

Related U.S. Application Data
(63) Continuation-in-part of application No. 10/620,740, filed on Jul. 16, 2003.
(63) Continuation-in-part of application No. 10/463,189, filed on Jun. 17, 2003.

(51) Int. Cl. A61B 17/22

(52) U.S. Cl. 606/159

(57) ABSTRACT

A flexible guidewire system for crossing an obstruction located in a patient's vessel comprising a flexible tubular casing slidable and rotatable over a pilot wire, the casing having an internal tubular wire-shield, at least a distal portion of the casing being a helical wire that is gated at its distal end, and a coupling means connected to the casing for rotating and linearly moving the casing and shield over the pilot wire.

### CROSS REFERENCE TO RELATED APPLICATIONS

This application is a continuation-in-part (CIP) of co-pending application Ser. No. 10/620740, filed on Jul. 16, 2003 (CT23), and also a CIP of another co-pending application Ser. No. 10/463189, filed on Jun. 17, 2003 (CT22).

All of the above are being incorporated herein by reference.

### BACKGROUND AND OBJECTIVES OF THE INVENTION

With age a large percentage of the population develops atherosclerotic and thrombotic obstructions resulting in partial or total occlusions of blood vessels in various parts of the human anatomy. Such obstructions are often treated with angioplasty or atherectomy catheters. A common preparatory step to such procedures is inserting a guidewire through the obstruction.

An objective of the present invention is to provide a simple and reliable flexible guidewire system capable of crossing tortuous vasculature and obstructions, particularly tight and total obstructions.

The above and other objectives of the invention will become apparent from the following discussion and the accompanying drawings.

### BRIEF DESCRIPTION OF THE FIGURES

FIG. 1 schematically shows a side view of a flexible guidewire system for crossing an obstruction in a vessel comprising a pilot wire over which a tubular casing with an internal tubular shield are slidable and rotatable through a coupling, being inserted at the patient's groin area, through his arterial system into his obstructed coronary artery;

FIGS. 2 and 3 show enlarged proximal and distal portions, respectively, of the guidewire system shown in FIG. 1;

FIG. 4 shows additional details of the guidewire system;

FIG. 5 shows a guidewire system where the distal end section of the tubular casing is pre-curved;

FIGS. 6, 7 and 8 show alternative flattened cross sections of wires that can be used to wind the casing;

FIG. 9 shows a modified distal end of a casing wherein the distal end of the shield is connected to a tube section through matching conical surfaces;

FIG. 10 shows an end view of the casing shown in FIG. 9;

FIG. 11 shows a modified distal end of a casing which is gated by a weld connecting the two most distal coils;

FIG. 12 shows an end view of the casing shown in FIG. 11;

FIG. 13 shows a partially cross sectioned view of a guidewire system in a flexible sleeve, with an inflatable chamber located at the distal end of the sleeve;

FIG. 14 shows a cross-sectional view along line 14-14 marked on FIG. 13;

FIG. 15 shows a partially cross sectioned view of a guidewire system in a flexible sleeve, with a selectively actuatable tongue at a distal end of said sleeve; and,

FIG. 16 shows a cross-sectional view along line 16-16 marked on FIG. 15.

### DETAILED DESCRIPTION OF THE DRAWINGS

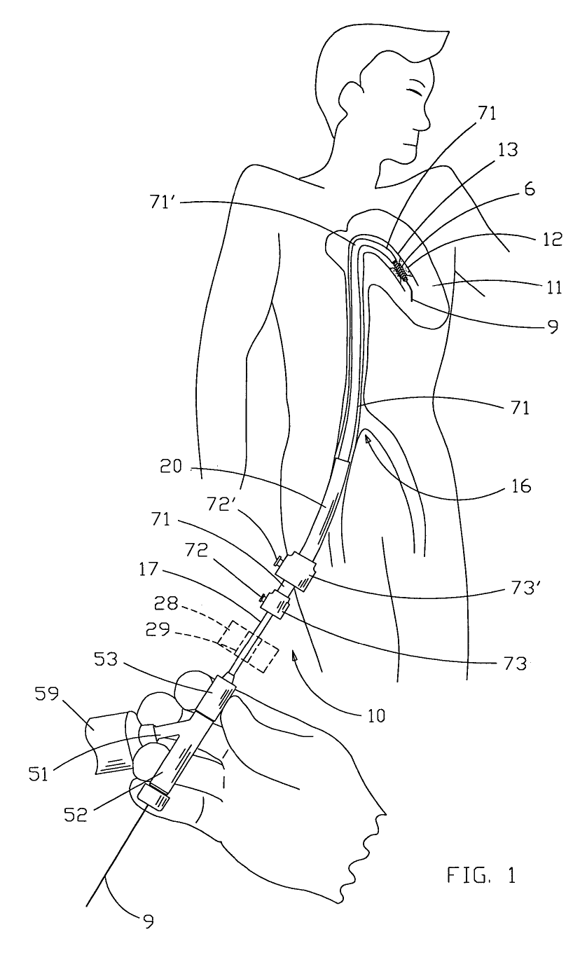

FIGS. 1, 2 and 3 show a flexible guidewire system 10 made of elongated components that are rotatable and slidable one relative to the other (the components' ends that go further into the vessel are referred to as “distal” and their other ends are referred to as “proximal”). The system is shown crossing an obstruction 12 located in a patient's coronary vessel 13 serving the heart 11 (the patient's anatomy and the system are illustrated schematically and are not drawn to scale).

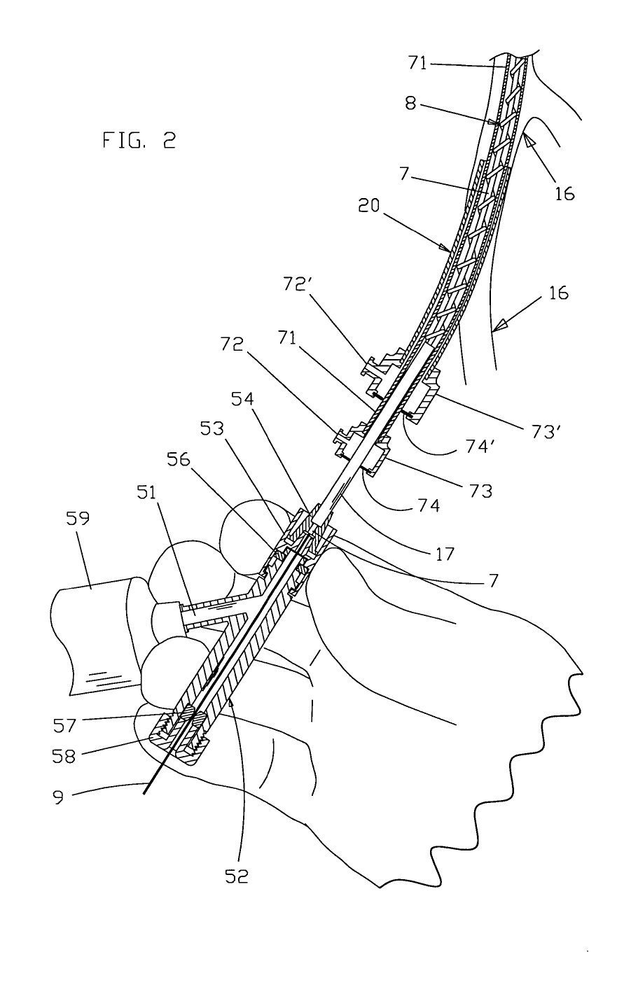

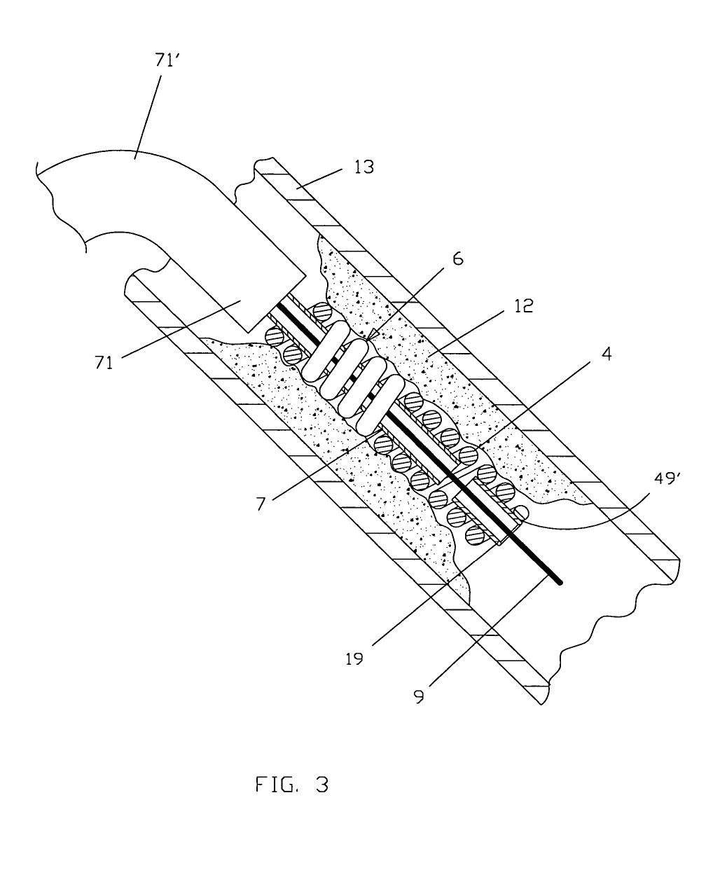

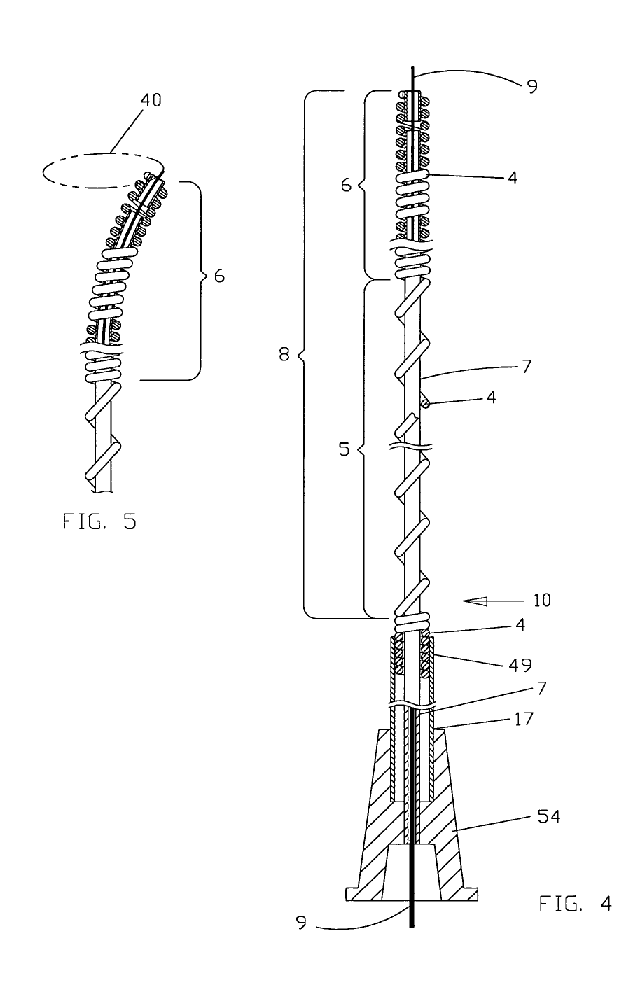

The system 10 comprises a flexible pilot wire 9, a flexible tubular casing 8 (note also FIG. 4) having an internal tubular pilot wire shield 7 disposed in and affixed to the casing. The casing and the shield are slidable and rotatable over the pilot wire. The flexible pilot wire can be a standard guidewire (guidewires are sold by numerous companies, e.g.: Boston Scientific, Natick, Mass.; Cook, Bloomington, Ind.). At least a distal portion 6 of the casing is a helical wire that is gated at its distal end by a tube section 19 that is secured to the helical wire by a weld 49′ (note FIG. 3). A coupling means, in the form of a tube 17 is connected to the casing by a weld 49 (note FIG. 4) for rotating and linearly moving the casing and the shield over the pilot wire.

The system 10 can be introduced into the patient's arterial system 16 through a flexible sleeve 71 that isolates it from the vessels' walls and directs the system to the obstruction site. An external port 72 is connected to the flexible sleeve through an annular chamber 73 that is attached to the proximal end of the sleeve. The chamber is equipped with a seal 74 that seals around a smooth outer surface of the tube 17. Optionally, the distal end section of the sleeve can be pre-curved, as shown in FIG. 1 and marked 71′, to direct the distal end of the system into a specific vessel and selectively bias it inside the vessel. The sleeve 71 can be inserted into the vasculature directly or through a standard introducer 20 having a port 72′, a chamber 73′ and a seal 74′ that seals on the outer surface of sleeve 71 (standard introducers are sold by numerous companies, e.g.: Boston Scientific, Natick, Mass.; Cook, Bloomington, Ind.).

The shield 7 has an open distal end and a proximal end that is connected to a rotating Y-connector 52, through its rotatable portion 53, that is a one part of a luer fitting (such rotating Y-connectors are sold by numerous companies, e.g.: EV3, Plymouth, Minn.). A second mating part of the luer fitting 54 is affixed (e.g., bonded) to both a proximal end of the tube 17 and to a proximal end of the shield 7. Thus, upon tightening the two parts of the luer fitting, the proximal end of the casing is coupled to the rotatable portion of the Y-connector through the tube 17 and the proximal end of the shield is also coupled to the rotatable portion of the Y-connector. The tightened luer fitting also hydraulically connects the shield to an external port 51 and a seal 56, incorporated in the rotary Y-connector, prevents leakage through the rotary connection.

At its proximal end the Y-connector is equipped with a compression-seal 57, the internal diameter of which decreases in response to tightening of a threaded cap 58 which reduces the length of the seal causing it to elastically deform and close the gap around the pilot wire 9, or in the absence of a pilot wire, to shut the proximal end of the Y-connector.

As illustrated in FIGS. 1 and 2, the system can be held by a single hand with the thumb and index finger imparting rotation to the luer fitting and thereby to the casing 8. An optional syringe 59, preferably with a small diameter piston suitable for generating higher pressures, is connected to the port 51 and can be used to deliver fluid (e.g., saline solution, radio-opaque fluid, drugs) through the distal end of the shield.

FIG. 3. shows the distal end of the system, wherein the distal portion of the casing is gated by the tube section 19 that is affixed to the casing by the weld 49′. A gap between the distal end of the shield 7 and the tube section 19 prevents interference between them when the casing is longitudinally compressed. The distal end of the wire 4 is ground down to form a smooth inclined plane to ease its penetration and minimize the likelihood of trauma to the vasculature 16 or to the vessel 13.

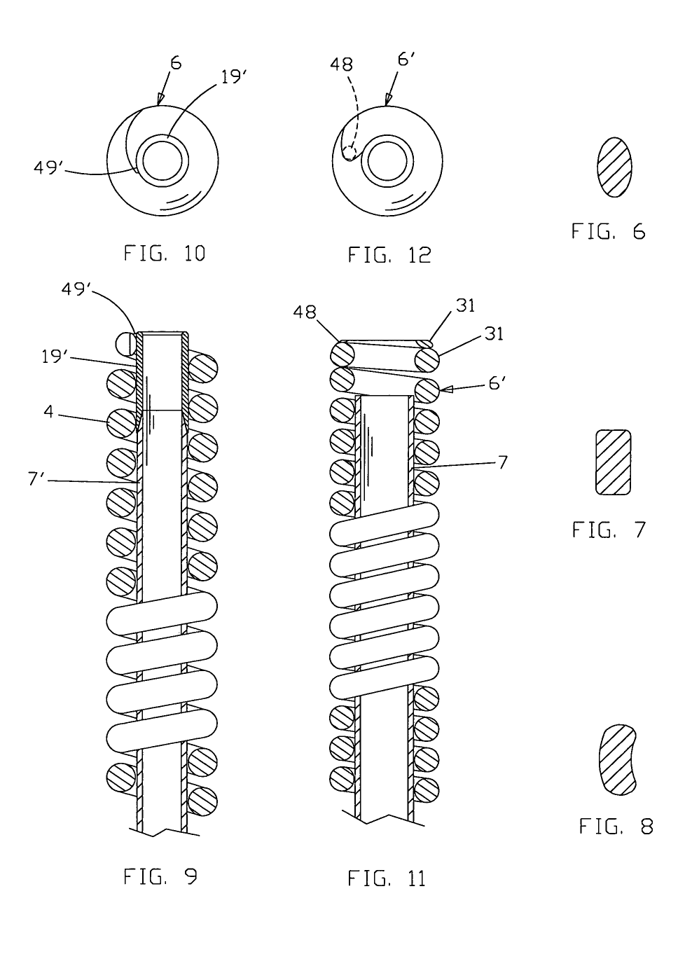

FIG. 4 shows further details of the casing 8 that is preferably made of a distal section 6 in the form of closely wound coils and a midsection 5 in the form of distantly spaced coils and both are wound from a continuous wire 4 for enhanced integrity. The closely wound coils provide enhanced flexibility whereas the distantly spaced coils provide enhanced torsional and longitudinal rigidity thereby reducing the elastic angular and linear deformation between the distal and proximal ends of the casing under torque and linear loading, respectively. As shown in FIGS. 3 and 4, the wire 4 has a round cross section, however, alternatively, the casing can be wound from a wire with a flattened cross-section, examples of which are shown in FIGS. 6, 7 and 8. The distal portion 6 can be relatively straight, as shown in FIG. 4. Alternatively, it can be pre-curved, as shown in FIG. 5, so that as the casing is rotated to start penetrating the obstruction 12, the distal tip moves along a circular path 40, increasing the probability that the distal tip would locate a soft point in a proximal end of the obstruction to start threading itself into. Once inside the obstruction, the pre-curvature can be altered by the surrounding obstruction to adapt to the trajectory of the distal end as it penetrates through the obstruction material.

The tube 17 is welded to the proximal end of the casing, and essentially serves as an extension of its proximal end with a smooth outside surface that provides a surface suitable for the seal 74 to seal against while the tube 17 is rotated and linearly moved through it. Alternatively, the system can be inserted directly through the introducer 20, in which case the seal 74′ provides the sealing around the tube 17. The casing can be driven (i.e., advanced and rotated), through the tube 17, by the physician's hand. Alternatively, a motor 28 (shown in FIG. 1 in intermittent line) can provide the rotation through its hollow output shaft 29 that is frictionally engaged with the tube 17, while the linear motion can be still provided by the a physician hand that holds the motor.

FIGS. 9 and 10 show a side view and an end view, respectively, of a modified distal end of a system wherein the distal end of the casing is gated by a tube section 19′ that is connected, through matching conical surfaces, to the shield 7′. As shown in FIG. 10, the distal end of the wire 4 is ground down to form a smooth inclined plane to ease its penetration and minimize the likelihood of trauma to the vasculature 16 or to the vessel 13.

FIGS. 11 and 12 show a side view and an end view, respectively, of a modified distal end of the system wherein the distal end of the flexible casing 6′ is gated by two distal coils 31 that are closely wound so that the spacing between them is smaller than the diameter of the pilot wire. Preferably, the coils are welded one to the other by a weld 48. The weld 48 also assures that fibrous material that may be encountered in the artery does not work its way in-between the coils when the casing is rotated and threaded through the obstruction. The distal end of the shield is slidably disposed inside the distal end of the casing so that it does not protrude out of it when the casing slightly compresses and shortens.

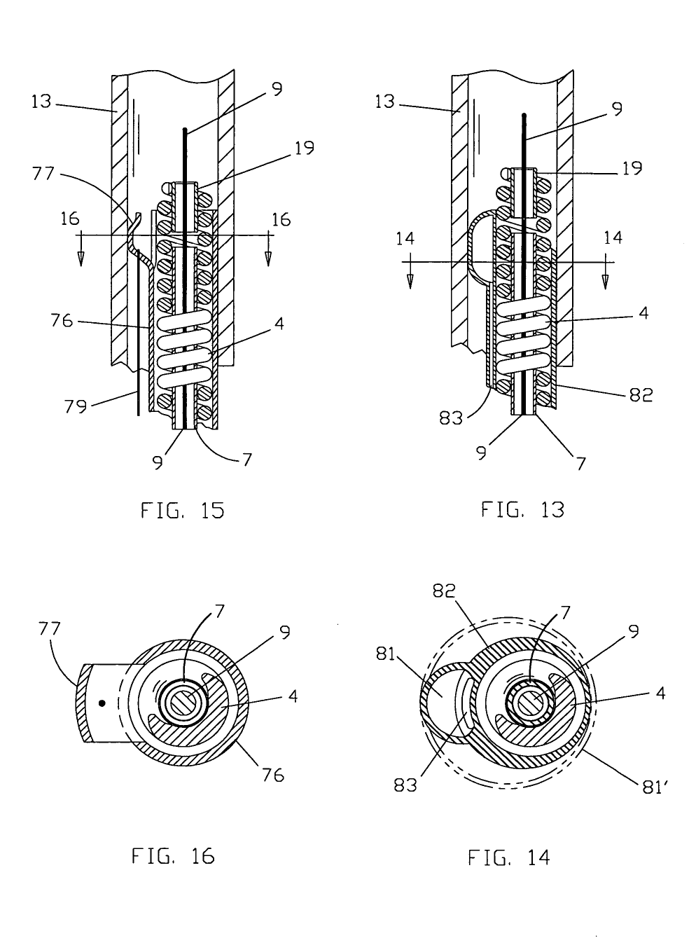

FIGS. 13 and 14 show cross-sectioned side and end views, respectively, of a biasing means in the form of an asymmetrical inflatable chamber 81 formed at the distal end of a flexible deflecting sleeve 82 which, when inflated through a channel 83 formed in the sleeve's wall, bears against the vessel's wall, eccentrically biasing the flexible sleeve in the vessel. When deflated, the chamber conforms to the sleeve to minimize interference with its insertion into the vessel. Alternatively, the chamber can be shaped as an asymmetrical toroidal inflatable chamber 81′ as shown in FIG. 14 by interrupted lines. This chamber, when inflated, establishes peripheral contact with the vessel's wall and thereby blocks blood flow between the sleeve and the vessel's wall, as well as eccentrically biases the sleeve (it can be understood that a symmetrical toroidal chamber can be provided for the purpose of blocking the flow around the sleeve while centering the biasing sleeve).

FIGS. 15 and 16 show cross-sectioned side and end views, respectively, of flexible sleeve 76 that has a tongue 77 which can be used to bias the sleeve in the vessel. The tongue can be energized against the vessel wall by pulling a flexible rope 79 and thereby moving the tongue from its relaxed position to the position shown in FIGS. 15 and 16.

### Operation

The previously described system, shown in FIGS. 1, 2 and 3, can be operated as following. The distal portion of the flexible pilot wire is inserted into a curved vessel, and assumes the vessel's geometry. Then the casing is inserted through the vasculature over the flexible pilot wire. The casing can be rotated to assist in its advancement over the pilot wire through curves of the vasculature while the flexible pilot wire guides the advancing casing. The rotation of the casing substantially reduces the longitudinal friction between the casing and the stationary pilot wire as well as longitudinal friction between the casing and its surroundings, i.e., the sleeve (assuming a sleeve is used) and the vessel or vessels through which the casing is advanced towards the obstruction. Further, if the casing in the form of a helical wire is turned in the direction that the coils are wound, the rotation generates a force that pulls and propels the casing forward through the vessels. Such pulling force generated at the distal end is significant because in order to deliver to the distal end the same amount of force through a tortuous path (as the path through the coronary vasculature commonly is), a larger push force would be required to be applied to the proximal end of the casing which may exceed the casing's columnar strength resulting in the casing buckling.

Once the casing is brought to the obstruction, the process of crossing an obstruction with a system according to the present invention can be done as follows:

Advancing the flexible pilot wire into the obstruction, preferably as far as it would go.

Inserting the casing to the obstruction and rotating it in the direction so that the helical wire propels and threads itself through the obstruction. In the process, the end of the helical wire may be advanced past the distal tip of the pilot wire and then the tip of the pilot wire may be advanced past the distal tip of the casing in a leapfrog-like manner. Once the pilot wire is advanced across the obstruction, the casing may be withdrawn, optionally by rotating it in the opposite direction to unthread it and to minimize longitudinal friction both with the pilot wire and with the surrounding casing, leaving the pilot wire in place for subsequent procedures such as angioplasty or atherectomy.

It is also possible to continue and rotate the casing, after it has been threaded across the obstruction, to increase the helical wire's proximal conveyance action, especially when working in an obstruction with a slurry-like consistency such as fresh blood clots.

The sequence of inserting the system's components into the vessel may be varied. Steps may be combined to streamline the procedure or added to improve it and to customize the procedure to the individual characteristics of an obstruction and its location and to the working preferences of the medical staff. For example, the system may be introduced percutaneously through a sleeve and/or an introducer or it may be introduced intra-operatively, i.e., by accessing vessel directly while it is exposed surgically. Additionally, a standard guiding catheter, either straight or curved, may be inserted into the vessel and used as a sleeve or biasing means to assist in positioning the system's components in the obstruction site. Further, the pilot wire and the casing can be pre-nested before they are inserted into the vessel.

Further, a system according to the present invention can have different diameters and lengths depending on the size and site of the vessel that it is intended for and on whether the system is to be used percutaneously or intra-operatively. For example, a system that is intended to be introduced percutaneously at the groin area for crossing an obstruction in a coronary vessel preferably utilizes as a pilot wire a standard guidewire with a 0.014″ (“denotes inches) diameter and a length of 120″ with a casing having an internal diameter of 0.020″, an outside diameter of 0.045″ and a length of 50″. The distal portion of the casing can be 10″ long, the midsection 30″ long and the tube 17 can be 10″ long. If the system utilizes a larger diameter pilot wire, such as an 0.035″ guidewire, the casing diameters can be increased accordingly. If the system is intended for use in peripheral (non-coronary) blood vessels or where direct access to the vessel is gained surgically (intraoperatively), the system can be shorter.

The above mentioned and other modifications and substitutions can be made in the system and in its operation within the spirit of the invention and the scope of the following claims.

1. A flexible guidewire system, for crossing an obstruction located in a patient's vessel, comprising:
a flexible pilot wire;
a flexible tubular casing having a tubular shield disposed in said casing, said casing and said shield being slidable and rotatable over said pilot wire, at least a distal portion of said casing being a helical wire that is gated at its distal end; and
a coupling means connected to said casing for rotating and linearly moving said casing and said shield over said pilot wire.

2. As in claim 1 wherein a proximal end of said shield is affixed to said casing.

3. As in claim 2 wherein said proximal end of said shield is hydraulically connected to an external port.

4. As in claim 1, wherein said distal end of said helical wire is gated by closely wound coils of said helical wire.

5. As in claim 4, wherein a distal end of said shield is slidably disposed in said helical wire.

6. As in claim 1, wherein said helical wire comprises a distal end that is gated by a tube section.

7. As in claim 6, wherein said distal end of said shield is connected to said tube section.

8. As in claim 1, wherein said casing has a midsection that comprises a helical wire with distantly spaced coils.

9. As in claim 8, wherein said distal portion of said casing and said midsection of said casing are wound of a continuous wire.

10. As in claim 1, wherein said distal portion of said casing is curved.

11. As in claim 1, wherein said flexible pilot wire is a standard guidewire.

12. As in claim 1, wherein said flexible guidewire system is disposed in a sleeve with a biasing means to deflect said casing in the vessel.

13. As in claim 12, wherein said sleeve comprises a pre-curved distal end section.

14. As in claim 12, wherein said sleeve comprises a selectively inflatable chamber formed at said distal end of said sleeve.

15. A process for crossing an obstruction in a patient's vessel comprising:
inserting through the vessel, to an obstruction, a flexible pilot wire;
advancing through the vessel over said pilot wire a flexible tubular casing containing an internal tubular pilot wire shield, at least a distal end of said casing being a helical wire that is gated at its distal end, and a proximal coupling means connected to said casing for rotating and linearly moving said casing and said shield over said pilot wire; and
threading said casing through the obstruction.

16. As in claim 15 wherein radio-opaque fluid is injected into said vessel through said distal end of said shield.

17. As in claim 15, wherein a portion of said pilot wire is inserted distally to said casing, into said vessel, thereby providing a lever arm to angularly align said casing with the vessel.

18. A process for crossing an obstruction in a patient's vessel comprising:
inserting through the vessel, to an obstruction, a flexible pilot wire;
advancing through the vessel over said pilot wire a flexible tubular casing containing an internal tubular shield, at least a distal end of said casing being a helical wire that is gated at its distal end, and a proximal coupling means connected to said casing for rotating and linearly moving said casing and said shield over said pilot wire;
advancing and rotating said casing, through said coupling means, beyond a distal tip of said pilot wire and threading it across the obstruction;
advancing said pilot wire across the obstruction; and
withdrawing said casing while leaving said pilot wire in the vessel.

19. As in claim 18 wherein fluid is injected through said distal end of said shield.

20. As in claim 18, wherein a portion of said pilot wire is inserted distally to said casing, into said vessel, thereby providing a lever arm to angularly align said casing with said vessel.

21. A process for crossing an obstruction in a patient's vessel comprising:
inserting into and advancing through the vessel a flexible tubular casing containing an internal tubular shield, at least a distal end of said casing being a helical wire that is gated at its distal end, and a proximal coupling means connected to said casing for rotating and linearly moving said casing and said shield;
advancing and rotating said casing, through said coupling means, thereby threading it across the obstruction;
inserting a pilot wire through said casing into the vessel and across the obstruction; and
withdrawing said casing while leaving said pilot wire in the vessel.

22. As in claim 21, comprising additionally a step of entering into the vessel a sleeve with a biasing means at its distal end, whereas said casing is entered into the vessel through said sleeve and said biasing means is used to deflect the position of said casing in said the vessel.

23. As in claim 22, wherein said sleeve comprising a selectively inflatable chamber formed at said distal end of said sleeve.
